(19) United States
(12) Patent Application Publication
(10) Pub. No.: US 2005/0202449 A1
(43) Pub. Date: Sep. 15, 2005

(54) Methods for detecting and assaying nucleic acid sequences using temperature cycling

(76) Inventors: Robert C. Getts, Collegeville, PA (US); James M. Kadushin, Gilbertsville, PA (US)

Correspondence Address: LAW OFFICE OF MORRIS E. COHEN, 1122 CONEY ISLAND AVENUE, SUITE 217, BROOKLYN, NY, 11230, US

(21) Appl. No.: 10/825,776

(22) Filed: Apr. 16, 2004

Related U.S. Application Data
(63) Continuation of application No. 10/050,088, filed on Jan. 14, 2002, now abandoned.
(60) Provisional application No. 60/261,231, filed on Jan. 13, 2001.

(51) Int. Cl. C12Q 1/68

(52) U.S. Cl. 435/6

(57) ABSTRACT

The present invention is directed to a method for determining the presence of a specific nucleotide sequence in a cDNA reagent of a target sample utilizing a capture reagent having at least one first arm containing a label capable of emitting a detectable signal and at least one second arm having a nucleotide sequence complementary to a capture sequence attached to the cDNA reagent on a microarray through temperature cycling.

### RELATED APPLICATIONS

The present application is a continuation of U.S. Nonprovisional application Ser. No. 10/050,088 filed Jan. 14, 2002 (pending) which claims the priority of U.S. Provisional application Ser. No. 60/261,231, filed Jan. 13, 2001. The contents of both of those applications are fully incorporated herein by reference.

### FIELD OF THE INVENTION

The present invention is related generally to nucleic acid assays, more particularly to methods for detecting and assaying nucleic acid sequences through the process of hybridization and temperature cycling.

### BACKGROUND OF THE INVENTION

Changes in gene expression patterns or in a DNA sequence can have profound effects on biological functions. Such variations in gene expression may result in altered physiological and pathological processes. Developing DNA technologies are providing rapid and cost-effective methods for identifying gene expression and genetic variations on a large-scale level. One useful development is the DNA microarray useful for rapidly detecting and assaying samples of target nucleic acid reagent. Each microarray is capable of performing the equivalent of thousands of individual “test tube” experiments over a short time period thereby providing rapid and simultaneous detection of thousands of expressed genes. Microarrays have been implemented in a range of applications such as analyzing a sample for the presence of gene variations or mutations (i.e. genotyping), or for patterns of gene expression.

Generally, a microarray comprises a substantially planar substrate such as a glass cover slide, a silicon plate or nylon membrane, coated with a grid of tiny spots or features of about 20 microns in diameter. Each spot or feature contains millions of copies of a specific sequence of nucleic acid extracted from a strand of deoxyribonucleic acid (DNA). Due to the number of features involved, a computer is typically used to keep track of each sequence located at each predetermined feature. Messenger RNA (mRNA) is extracted from a sample of cells. The mRNA serving as a template, is reverse transcribed to yield a complementary DNA (cDNA). As a first example of the prior art techniques, one or more labels or markers such as fluorescence are directly incorporated into the copies of cDNA during the reverse transcription process. The labeled copies of cDNA are broken up into short fragments and washed over the microarray. Under suitable hybridization conditions, the labeled fragments are hybridized or coupled with complementary nucleic acid sequences (i.e. gene probes) attached to the features of the microarray for ready detection thereof. This labeling method has been commonly referred to as “direct incorporation”.

Upon hybridization of the cDNA to the microarray, a detectable signal (e.g. fluorescence) is emitted for a positive outcome from each feature containing a cDNA fragment hybridized with a complementary gene probe attached thereto. The detectable signal is visible to an appropriate sensor device or microscope, and may then be detected by the computer or user to generate a hybridization pattern. Since the nucleic acid sequence at each feature is known, any positive outcome (i.e. signal generation) at a particular feature indicates the presence of the complementary cDNA sequence in the sample cell. Although there are occasional mismatches, the attachment of millions of gene probes at each spot or feature ensures that the detectable signal is strongly emitted only if the complementary cDNA of the test sample is present.

A second example of a prior art method of preparing a target nucleic acid reagent for detection and assay by a microarray is shown in FIG. 1 and described as follows. Using known methods, a plurality of gene probes consisting of known nucleic acid sequences are each affixed or printed at a predetermined location on the surface of a microarray. The attachment of the gene probe to the microarray is typically accomplished through known robotic or laser lithographic processes. The sample can be extracted from cells of organisms in the form of RNA.

Since RNA is relatively unstable and decomposes rapidly and easily, a more stable and resistant form of nucleic acid is typically used. The stable nucleic acid is complementary DNA which is prepared from the RNA sample (e.g. total RNA and poly(A)⁺RNA) through conventional techniques for implementing reverse transcription. Reverse transcriptase and reverse transcription primers (RT primers) having a capture sequence attached thereto, are used to initiate the reverse transcription process. This results in the formation of the target cDNA with the capture sequence located at the 5′ end. The newly formed target EDNA with the capture sequence is then isolated from the mRNA sample and precipitated. The target cDNA is hybridized to the complementary gene probes affixed to the microarray. After the target cDNA and the microarray are hybridized, the microarray is washed to remove any excess RT primers prior to labeling. A mixture containing labeled “dendritic nucleic acid molecules”, or “dendrimers”, is then prepared.

Dendrimers are complex, highly branched molecules, and are comprised of a plurality of interconnected natural or synthetic monomeric subunits of double-stranded DNA forming into stable spherical-like core structures with a predetermined number of “free ends” or “arms” extending therefrom. Dendrimers provide efficient means for labeling reactions such as fluorescence, for example, and facilitate direct calculations of the number of transcripts bound due to their predetermined signal generation intensity and proportional relationship to the bound cDNA on the microarray.

Each dendrimer includes two types of hybridization “free ends” or “arms” extending from the core surface. Each dendrimer may be configured to include at least one hundred arms of each type. The arms are each composed of a single-stranded DNA of a specific sequence that can be ligated or hybridized to a functional molecule such as a target or a label. The target molecule can be attached to one type of arm to provide the dendrimer with targeting capabilities, and the label molecule can be attached to the other type of arm to provide the dendrimer with signal emission capabilities for detection thereof. The targeting molecule is typically an oligonucleotide that is complementary to the capture sequence of a target, and the label molecule is typically an oligonucleotide linked to a label or marker. Using simple DNA labeling, hybridization, and ligation reactions, a dendrimer can thus be configured to act as a highly labeled, target specific probe, and therefore may be used in a microarray system for DNA analysis. Dendrimers are described in greater detail in U.S. Pat. Nos. 5,175,270 and 5,484,904, in Nilsen et al., Dendritic Nucleic Acid Structures, J. Theor. Biol., 187, 273-284 (1997); and in Stears et al., A Novel, Sensitive Detection System for High-Density Microarrays Using Dendrimer Technology, Physiol. Genomics, 3:93-99 (2000), the entire content of each are incorporated herein by reference.

The prepared mixture is formulated in the presence of a suitable buffer to yield a dendrimer hybridization mixture containing dendrimers with labels attached to one type of arms, and with oligonucleotides complementary to the capture sequences of the cDNA attached to the other type of arms. The labeled dendrimers are added to the microarray for hybridization with the capture sequences of the bound cDNA to generate a detectable signal from the corresponding feature. The microarray is washed to remove any excess unhybridized dendrimer molecules to reduce unwanted noise generation. The microarray is scanned using conventional techniques to detect the detectable signal emitted by the labels to generate a particular hybridization pattern for analysis. It is known that the above-described prior art methods require significant time, effort and labor in the preparation and assay of the sample including the hybridization and washing steps. The typical time required to process and assay the sample can extend to at least two days.

It would be highly desirable to substantially reduce the amount of time and the number of steps required for preparing a sample and performing the assay without sacrificing desirable attributes such as sensitivity, low background “noise”, and minimal “false positives”. It would be a significant advance in gene expression detection microarrays to further provide a method that significantly reduces the complexity and the labor needed to prepare the gene samples and conduct the assay which can be carried out using conventional laboratory reagents, equipment and techniques.

### SUMMARY OF THE INVENTION

It is an object of the present invention to reduce the number of steps necessary to produce a detection array.

It is a further object of the present invention to reduce the time required to produce a detection array.

It is a further object of the present invention to provide a method for producing a detection array in which cDNA and dendrimer are both applied to the array concurrently.

Further objects, advantages and features of the invention will become apparent in conjunction with the detailed disclosure provided herein.

In accordance with the present invention, methods are provided for improving the production of a microarray.

The present invention relates generally to methods for assaying and detecting the presence of a specific sequence of nucleotides in a nucleic acid target molecule of a sample through the process of hybridization. The present invention significantly reduces the time and labor that are typically required to process and assay the nucleic acid target molecule for obtaining information about the genetic profile of the target nucleic acid reagent and the source from which the sample was obtained. The present invention further provides a microarray with excellent sensitivity and low background “noise”, and minimal “false positives”. The method of the present invention may be used in a range of genomic applications such as gene expression profiling and high-throughput functional genomic analysis.

The methods of the invention are designed to significantly reduce the steps and time required for producing a detection array and for determining the presence of at least one specific nucleotide sequence in a target nucleic acid (e.g. obtained from a target biological sample). In addition, procedures are provided which require less reverse transcription (RT) primer than normally required to prepare the target nucleic acid reagent, incorporate the use of a spin column to prepare the target nucleic acid reagent, do not require the use of a cDNA buffer, and/or do not require competitor DNA.

The method of the present invention generally comprises contacting both a target nucleic acid reagent and a capture reagent coupled to a label molecule to a microarray comprising a plurality of gene probes, and treating the microarray to induce the target nucleic acid reagent to first hybridize with the complementary sequences of the gene probes on the microarray, and then to induce the capture reagent to hybridize with the gene probe-hybridized target nucleic acid reagent. The advantages that are offered by this procedure are designed to improve the kinetics of hybridization of each of the two components, i.e. target nucleic acid to probe, and capture reagent to target nucleic acid.

In accordance with a preferred embodiment of the invention, both cDNA and a dendrimer are concurrently applied to a microarray under conditions designed to ensure that the cDNA will initially hybridize only to the microarray, but not to capture sequences on the dendrimer. Subsequently, the conditions are modified to allow the cDNA will hybridize to the dendrimer.

In a further preferred embodiment, temperature cycling is used to selectively control hybridization between the target nucleic acid and the microarray, and hybridization between the capture reagent and the microarray (preferably cDNA—microarray hybridization and cDNA—dendrimer hybridization, respectively). By using such cycling, hybridization can be carefully controlled such that cDNA initially hybrizides only to the microarray, with subsequent hybridization of cDNA to the dendrimer.

In one such embodiment, a temperature “cycling down” is conducted, from a higher temperature for the initial target nucleic acid—microarray hybridization, to a lower temperature for the subsequent capture reagent—target nucleic acid hybridization. In an alternate embodiment, a temperature “cycling up” is conducted. In this embodiment, a blocking oligonucleotide (“the blocker”) is used to block the capture sequence complement on the capture reagent to yield a blocked capture reagent which cannot hybridize with the target nucleic acid. (Or, similarly, the blocker can be hybridized to the capture sequence of the target nucleic acid, to yield a blocked target nucleic acid and prevent hybridization between the target nucleic acid and the capture reagent). The initial temperature for target nucleic acid hybrization to the microarray is selected to be a temperature which is lower than the melting temperature for the blocking oligonucleotide. Subsequently, the temperature is raised to a temperature above the melting temperature for that blocking oligonucleotide, causing dissociation of the blocker from the capture reagent, and allowing capture reagent hybridization to the target nucleic acid sequence.

In further embodiments, each of the methods described can be used with or without a spin column to prepare the target nucleic acid reagent.

In one particular aspect of the present invention, there is provided a method for determining the presence of at least one specific nucleotide sequence in a target nucleic acid reagent obtained from a target biological sample. The method comprises the steps of:

(a) contacting a microarray with:
(i) at least one specific nucleotide sequence of the target nucleic acid reagent, each of said at least one nucleotide sequence further including a capture sequence, and
(ii) a capture reagent having at least one first arm containing a label capable of emitting a detectable signal and at least one second arm having a nucleotide sequence complementary to the capture sequence of the at least one specific nucleotide sequence,
   said microarray having thereon a plurality of features, each of said plurality of features including a probe nucleotide sequence; and

(b) treating the microarray from step (a) at a temperature and for a time sufficient to induce the at least one specific nucleotide sequence to hybridize with the probe nucleotide sequence complementary thereto on the microarray, and then to induce the capture reagent to hybridize to the capture sequence of the at least one specific nucleotide sequence hybridized to the microarray wherein the presence of the latter hybridization results in the emission of the detectable signal from the corresponding feature, and the absence thereof results in no emission of the detectable signal from the corresponding feature, thus generating a detectable hybridization pattern for subsequent analysis.

In a preferred form of the invention, the capture reagent is a dendrimer.

### BRIEF DESCRIPTION OF THE DRAWINGS AND THE PREFERRED EMBODIMENTS

The following drawings in which like reference characters indicate like parts are illustrative of embodiments of the invention and are not to be construed as limiting the invention as encompassed by the claims forming part of the application.

FIG. 1 is a schematic representation of prior art steps for preparing and assaying a target nucleic acid reagent on a microarray;

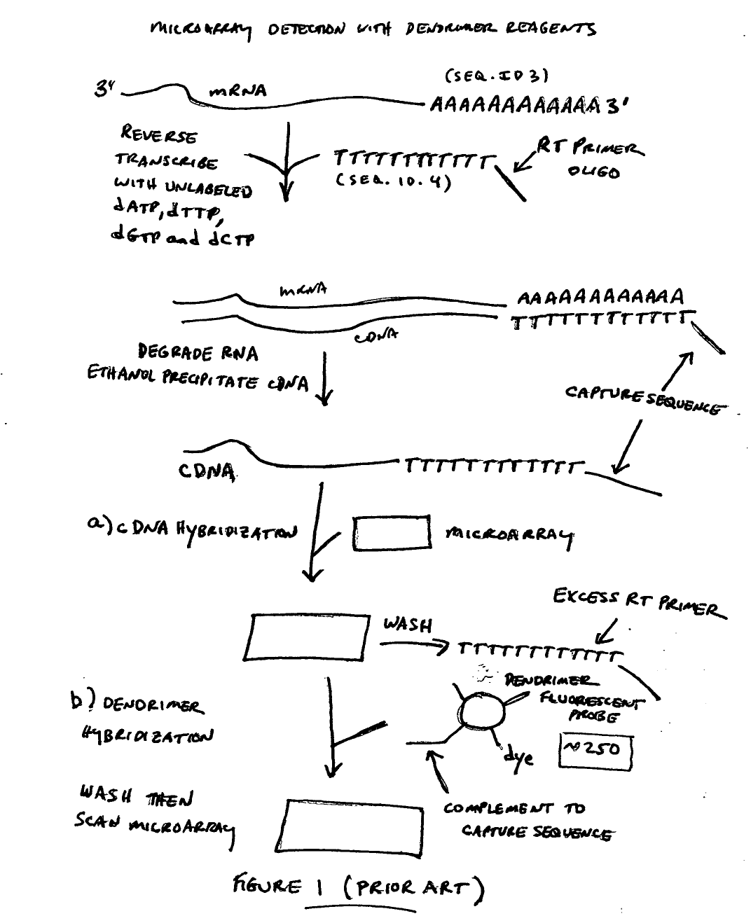

FIG. 2 is a schematic representation of a method for preparing and assaying a target nucleic acid reagent on a microarray in one embodiment of the present invention;

FIG. 3 is a schematic representation of a method for preparing and assaying a target nucleic acid reagent on a microarray for a second embodiment of the present invention; and

FIG. 4 is a cross sectional view of a spin column assembly used in accordance with the method represented in FIG. 3;

FIG. 5 is a schematic representation of a method for preparing and assaying a target nucleic acid reagent on a microarray for a third embodiment of the present invention; and

FIG. 6 is a schematic representation of a method for preparing and assaying a target nucleic acid reagent on a microarray for a fourth embodiment of the present invention.

### DETAILED DESCRIPTION OF THE INVENTION

The present invention is generally directed to a method for preparing a target nucleic acid reagent comprising a sequence of nucleotides for detection and assay on a microarray in a manner that significantly reduces the time and effort typically required in assaying a genomic sample on a microarray. The method of the present invention provides the advantage of preparing the target nucleic acid reagent in shorter period of time, using fewer steps but providing the sensitivity, low background “noise”, and minimal “false positives” required for laboratory and clinical use. The cost effective and efficient manner by which the target nucleic acid reagent is prepared and by which the method of the present invention can be implemented using conventional laboratory techniques, equipment and reagents, makes the present invention especially suitable for use in genomic applications such as gene expression profiling and high-throughput functional genomic analysis. The term “target nucleic acid reagent” as used herein is meant to encompass any DNA or RNA-based genetic material processed or extracted from a natural source for assay on a microarray.

Before the present invention is further described, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In the methods of the present invention, an array of DNA or gene probes fixed or stably associated with the surface of a substantially planar substrate is prepared as conventionally known in the art. A variety of different microarrays that may be used are known in the art. The substrates with which the gene probes are stably associated may be fabricated from a variety of materials, including plastic, ceramic, metal, gel, membrane, glass, and the like. The microarrays may be produced according to any convenient methodology, such as pre-forming the gene probes and then stably associating them with the surface of the support or growing the gene probes directly on the support. A number of different microarray configurations and methods for their production are known to those of skill in the art, as described, for example, in Science, 283, 83, 1999, the content of which is incorporated herein by reference.

The DNA or gene probes of the microarrays which are capable of sequence specific hybridization with a target nucleic acid reagent extracted from a target sample of cells, may be comprised of polynucleotides or hybridizing analogues or mimetics thereof, including, but not limited to, nucleic acid in which the phosphodiester linkage has been replaced with a substitute linkage group, such as phosphorothioate, methylimino, methylphosphonate, phosphoramidate, guanidine and the like, nucleic acid in which the ribose subunit has been substituted, e.g. hexose phosphodiester; peptide nucleic acid, and the like. The length of the gene probes will generally range from 10 to 1000 nucleotides. In the preferred embodiment, the DNA or gene probes are each arranged or sequenced for hybridization with the target nucleic acid reagent comprising DNA, more preferably cDNA from the gene of concern.

In some embodiments of the invention, the gene probes will be oligonucleotides having from 15 to 150 nucleotides and more usually from 15 to 100 nucleotides. In other embodiments the gene probes will be longer, usually ranging in length from 150 to 1000 nucleotides, where the polynucleotide probes may be single or double stranded, usually single stranded, and may be PCR fragments amplified from cDNA, cloned genes, or other suitable sources of nucleic acid sequences. The DNA or gene probes on the surface of the substrates will preferably correspond to, but are not limited to, known genes of the physiological source being analyzed and be positioned on the microarray at a known location so that positive hybridization events may be correlated to expression of a particular gene in the physiological source from which the target nucleic acid reagent is derived. Because of the manner in which the target nucleic acid reagent is generated preferably in the form of DNA, as herein described below, the microarrays of gene probes will generally have sequences that are complementary to the DNA-based strands, including but not limited to, cDNA strands, of the gene to which they correspond.

The term “label” is used herein in a broad sense to refer to agents that are capable of providing a detectable signal, either directly or through interaction with one or more additional members of a signal producing system. Labels that are directly detectable and may find use in the present invention include, for example, fluorescent labels such as fluorescein, rhodamine, BODIPY, cyanine dyes (e.g. from Amersham Pharmacia), Alexa dyes (e.g. from Molecular Probes, Inc.), fluorescent dye phosphoramidites, and the like; and radioactive isotopes, such as ³² S, ³² P, ³ H, etc.; and the like. Examples of labels that provide a detectable signal through interaction with one or more additional members of a signal producing system include capture moieties that specifically bind to complementary binding pair members, where the complementary binding pair members comprise a directly detectable label moiety, such as a fluorescent moiety as described above. The label is one that preferably does not provide a variable signal, but instead provides a constant and reproducible signal over a given period of time.

The present invention further utilizes a capture reagent which is composed of at least one first arm containing a label capable of emitting a detectable signal and at least one second arm having a nucleotide sequence complementary to a capture sequence attached to the target nucleic acid such as DNA, for example. One such example is a “dendritic nucleic acid molecule”, or “dendrimer”. Briefly, dendrimers are complex, highly branched molecules, and are comprised of a plurality of interconnected natural or synthetic monomeric subunits of double-stranded DNA forming into stable, spherical-like core structures with a pre-determined number of “arms” or “free ends” extending therefrom for the purposes descried herein. Typically, the capture reagent will have multiple, typically many first and second arms. Besides dendrimers, carbohydrates, proteins, nucleic acids, and the like may be used as the capture reagent. Dendrimers will be described hereinafter as illustrative of suitable capture reagents.

Each dendrimer may be configured to include two types of hybridization “free ends” or “arms” extending from the core surface. Each dendrimer may be configured to include at least one hundred arms of each type. The arms are each composed of a single-stranded DNA of a specific sequence that can be ligated or hybridized to a functional molecule such as a target or a label. The target molecule can be attached to one type of arm to provide the dendrimer with targeting capabilities, and the label molecule can be attached to the other type of arm to provide the dendrimer with signal generation capabilities for detection. The targeting molecule is typically an oligonucleotide that is complementary to the capture sequence of the target nucleic acid reagent, and the label molecule is typically an oligonucleotide linked to a label or marker. Using simple DNA labeling, hybridization, and ligation reactions, a dendrimer may be configured to act as a highly labeled, target specific probe molecule, and therefore may be used in a microarray system for DNA analysis.

A dendrimer commonly used in the art may be obtained from the product 3DNA™ expression array reagent which is available from Genisphere Inc. and Datascope Corp. of Montvale, N.J. The application of the 3DNA™ reagent, is relatively straightforward. 3DNA™ reagent is available with either Cy3™ or Cy5™ labels attached thereto, making possible either single or dual channel detection in microarray assays. The labeled 3DNA™ capture reagent further may be adapted to include a “capture sequence” that is complementary to the 5′ end of a RT primer used to produce the target nucleic acid reagent which enables the capture reagent to hybridize to target nucleic acid reagent under suitable conditions during assay.

The labeled 3DNA™ capture reagent provides a more intense, predictable and consistent signal than the direct incorporation method described above, for two reasons. First, since the fluorescent dye is part of the 3DNA™ capture reagent, it does not have to be incorporated during the preparation of the target nucleic acid reagent (e.g., cDNA), thus avoiding the inefficient and unpredictable enzymatic incorporation of fluorescent dye nucleotide conjugates into the reverse transcript. Second, because each 3DNA™ capture reagent contains an average of about 250 or more fluorescent dyes and each target nucleic acid hybridized to the microarray can be readily detected by a single 3DNA™ capture reagent, the signal generated from each message will be largely independent of base composition or length of the corresponding transcript.

Further information regarding the structure, configuration and production of dendrimers is also disclosed in U.S. Pat. Nos. 5,175,270, 5,484,904, and 5,487,973, the contents of each are incorporated herein by reference.

In accordance with one embodiment of the present invention, a target nucleic acid, preferably in the form of a cDNA, prepared from a biological sample, and a capture reagent, preferably in the form of a dendrimer, are concurrently contacted with a microarray comprising a plurality of gene probes. The microarray is then treated at a temperature and for a time sufficient to induce hybridization between the target nucleic acid reagent and the complementary gene probes, and thereafter induce the capture reagent to hybridize with the target nucleic acid reagent, whereupon a detectable signal may be generated to render the particular hybridization pattern visible.

For example, this concurrent contact may be made individually with each reagent being applied to the microarray relatively simultaneously, and then allowing the components to mix on the microarray. Or, in an alternate embodiment, the target nucleic acid, preferably in the form of a cDNA, prepared from a biological sample, and the capture reagent, preferably in the form of a dendrimer, are mixed to yield a mixture. This mixture is then contacted with the microarray comprising a plurality of gene probes.

In a preferred embodiment, the method further comprises cycling the temperature of the microarray to selectively hybridize the target nucleic acid reagent with the microarray, and thereafter hybridize the capture reagent with the target nucleic acid reagent. In this manner, the cycling of the temperature provides precise control of the selectivity and the ordering of the hybridization processes, thus enabling the reduction in the number of process steps and time required for carrying out the assay.

It is noted that the hybridization between the target nucleic acid reagent and the microarray, and between the target nucleic acid reagent and the capture reagent (e.g. dendrimer) may be carried out in any suitable order. The capture reagent (e.g. dendrimer) is labeled and thus capable of generating the same signal of known intensity, thus each positive signal in the microarray can be “counted” in order to obtain quantitative information about the genetic profile of the target nucleic acid reagent.

In one embodiment of the present invention, fluorescent labeled dendrimers may be prepared by ligating a nucleic acid sequence or strand complementary to the capture sequence of a target nucleic acid reagent to the purified dendritic core material as prepared by the previously described methods (see Nilson et al., and Stears et al., supra; and the '270, '904, and '973 patent citations as previously mentioned). Labeled dendrimers ligated with the capture sequence, are able to target and hybridize with a target nucleic acid reagent such as a cDNA with a specific capture sequence attached thereto.

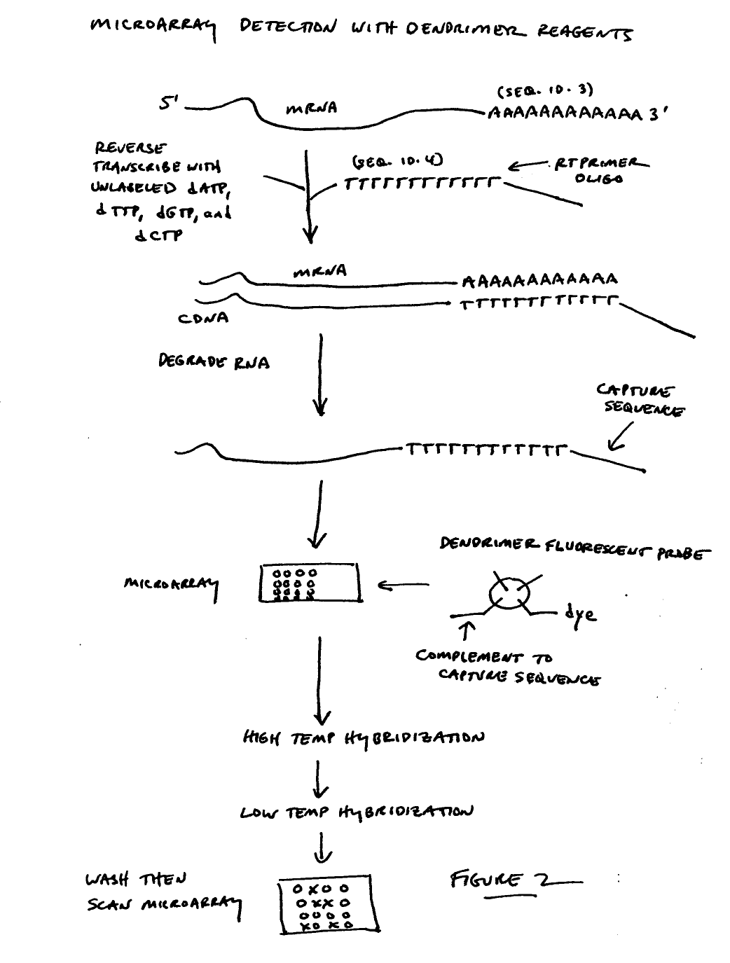

FIG. 2 illustrates in greater detail the present method in accordance with principles of the present invention. The target nucleic acid reagent for use in determining the genomic information of a sample is generally prepared from a RNA that is derived from a naturally occurring source. The RNA may be selected from total RNA, poly(A)⁺RNA, amplified RNA and the like. The initial RNA source may be present in a variety of different samples, and can be derived from a physiological source. The physiological source may be derived from a variety of eukaryotic sources, with physiological sources of interest including sources derived from single celled organisms such as yeast and multi-cellular organisms, including plants and animals, particularly mammals, where the physiological sources from multi-cellular organisms may be derived from particular organs or tissues of the multi-cellular organism, or from isolated cells derived therefrom.

In obtaining the RNA for processing and analysis, the physiological source may be subjected to a number of different processing steps, where such known processing steps may include tissue homogenation, cell isolation and cytoplasmic extraction, nucleic acid extraction and the like. Methods of isolating RNA from cells, tissues, organs or whole organisms are known to those of ordinary skill in the art and are described, for example, in Maniatis et al., Molecular Cloning: A Laboratory Manual, 2^{nd} ed., Cold Spring Harbor Laboratory Press, 1989, and in Ausubel et al., Current Protocols in Molecular Biology, John Wiley & Sons, Inc., 1998, the content of each are incorporated herein by reference.

In a preferred embodiment of the present invention, the extracted RNA is a polyadenylated RNA (poly(A)⁺RNA). The poly(A)⁺RNA includes an oligonucleotide which is comprised of a strand of adenine bases, or poly dA sequence, and provides a hybridization site for reverse transcription primers having a complementary oligonucleotide which is comprised of a strand of thyrnine bases, or poly dT sequence. This facilitates the attachment of the reverse transcription primers at appropriate sites to initiate the process of reverse transcription for forming the target nucleic acid reagent (e.g., cDNA) under suitable conditions.

It is noted that poly(A)⁺RNA is typically present in most genomic samples and in all genomic samples of mammalian origin such as from humans, mice, rats, pigs and the like. The present invention may also be used in conjunction with non-poly(A)⁺RNA samples as well. Such non-poly(A)⁺RNA lacks a poly dA sequence useful as an attachment site for the RT primers. Accordingly, such non-poly(A)⁺RNA is prepared by attaching or ligating a suitable attachment polynucleotide complementary to the RT primers used for facilitating reverse transcription.

Referring back to FIG. 2, the RT primer possessing a poly(dT) sequence and a capture sequence 5′ end, attaches to the complementary polyadenylated 3′ end of the mRNA sample. Reverse transcription is initiated in the presence of reverse transcriptase and deoxynucleotide triphosphates (i.e., dATP, dTTP, dGTP and dCTP). The mRNA is purged through suitable means including ethanol precipitation to yield a single stranded DNA or complementary DNA (cDNA), a target nucleic acid reagent. The polythymylated 5′ end of the cDNA inherits the capture sequence attached to the RT primer.

In one preferred example, the RT primers may be obtained from Genisphere, Inc. The nucleotide sequences of the primers corresponding to Cy3™ and Cy5™ are:

Cy3
5′-GGC CGA CTC ACT GCG CGT CTT CTG TCC CGC C-
oligo dT17-3′;
and
Cy5
5′-CCT GTT GCT CTA TTT CCC GTG CCG CTC CGG T-
oligo dT17-3′.

It is noted that these sequences can be found in Genisphere, Inc. protocols for their gene expression detection kits. The complement of the capture sequences are found on the fluor labeled capture reagents, or dendrimers. Although the present example is described in combination with use of Cy3™ and Cy5™, practically any fluor can be used, including, but not limited to, Alexa Fluors™ and other labeling dyes available from Molecular Probes, Inc. of Eugene, Oreg.

The capture reagent (preferably a dendrimer), coupled to an oligonucleotide (“complement”) complementary to the capture sequence of the target nucleic acid reagent, is added to the target nucleic acid reagent (preferably cDNA) to yield a hybridization mixture. The capture sequences and the complementary oligonucleotide have sufficient base units to irreversibly hybridize under suitable conditions including time and temperature sufficient for promoting the hybridization of the dendrimer to the target nucleic acid reagent (cDNA) as known by those of ordinary skill in the art. Suitable hybridization conditions are disclosed in Maniatis et al., where conditions may be modulated to achieve a desired specificity in hybridization. It is further noted that any suitable hybridization buffers may be used in the present invention.

The components (i.e., capture reagent and target nucleic acid reagent) of the hybridization mixture is then contacted with a microarray comprising multiple features each containing a specific nucleic acid sequence (typically in the form of a fragment of a cDNA, although any source for the nucleic acid sequences may be utilized). As noted, the method of the present invention; also encompasses applying the capture reagent and the target nucleic acid reagent (cDNA) to the microarray to yield the hybridization mixture upon contact.

In one preferred form of the invention, the hybridization mixture and the microarray is incubated at a first temperature, preferably in the range of from about 65° to 75° C., for a sufficient time, preferably overnight, to allow the target nucleic acid reagent (cDNA) to hybridize with the complementary nucleic acid sequence (i.e., gene probe) of the corresponding microarray feature. After the overnight hybridization is completed, the temperature is rapidly cycled down to a lower second temperature in the range of from about 50° to 55° C. The microarray and mixture is incubated at the lower second temperature for a sufficient time, preferably from about 4 to 6 hours, to allow the capture reagent to hybridize with the capture sequence of the target nucleic acid reagent (e.g., cDNA). The temperatures disclosed above may be adjusted in order to suit the requirements necessary to ensure complete hybridization to a selected microarray and to a selected capture reagent, which both can be suitably determined by those of ordinary skill in the art.

Any excess capture reagent present after the hybridization can undesirably interfere with the signal detection of the hybridized microarray and the resulting hybridization pattern, and is thus preferably removed during the processing and assay. Following the hybridization step, an optional washing step may be employed to purge the non-hybridized complexes from the inicroarray, thus leaving behind a visible, discrete pattern of hybridized cDNA-dendrimers bound to the microarray. To accomplish this, the microarray containing the hybridized cDNA sample, may optionally be washed with buffer solutions selected from sodium dodecyl sulfate (SDS) and standard saline citrate (SSC), to remove any of the excess capture reagent that may be present. A variety of wash solutions and protocols for their use are known to those of skill in the art and may be used. The specific wash conditions employed will necessarily depend on the specific nature of the signal producing system that is employed, and will be known to those of skill in the art familiar with the particular signal producing system employed.

Following hybridization and any washing step(s) and/or subsequent treatments, as described above, the resultant hybridization pattern is detected through a suitable commercially available microarray scanner. In detecting or visualizing the hybridization pattern, the intensity or signal value of the label may be qualitatively and/or quantitatively detected.

The resultant hybridization pattern of labeled target nucleic acid reagent (e.g., cDNA) may be visualized or detected in a variety of ways, with the particular manner of detection being chosen based on the particular label of the dendrimer used in the present invention, where representative detection systems include scintillation counting, autoradiography, fluorescence measurement, calorimetric measurement, light emission measurement, or so forth.

Following detection or visualization, the hybridization pattern can be used to determine qualitative and/or quantitative information about the genetic profile of the labeled target nucleic acid reagent that was contacted with the microarray to generate the hybridization pattern, as well as the physiological source from which the labeled target nucleic acid reagent was derived. From this data, one can also derive information such as the types of genes expressed in the tissue or cell that is the physiological source, as well as the levels of expression of each gene. Where one uses the subject methods in comparing target nucleic acid reagent from two or more physiological sources, the hybridization patterns may be compared to identify differences between the patterns. With microarrays in which each of the different probes corresponds to a known gene is employed, any discrepancies can be related to a differential expression of a particular gene in the physiological sources being compared. Thus, the subject methods find use in differential gene expression assays, where one may use the subject methods in the differential expression analysis of: diseased and normal tissue, e.g. neoplastic and normal tissue; different tissue or subtissue types; or so forth.

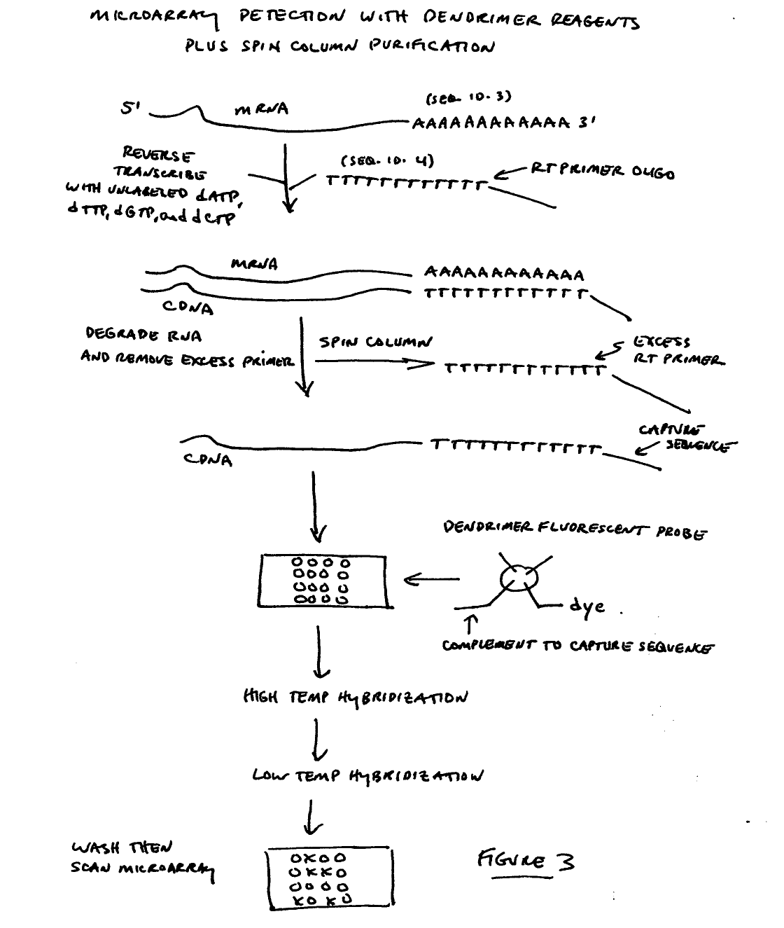

Referring to FIG. 3, a schematic representation of a method of the present invention is illustrated for an alternate embodiment of the present invention. The steps of the method are essentially the same as the method described in FIG. 2 except for the addition of an optional purification step utilizing a spin column to purify the newly formed target nucleic acid reagent (e.g., cDNA) prior to its application to the microarray. The spin column of purification step serves to removes any excess RT primers, which may still remain prior to application of the cDNA to the microarray. The prior removal of excess RT primers ensures that the capture reagent is utilized efficiently providing a strong signal emission. Spin columns are known devices used to separate one or more components from a mixture through the use of centrifugal force. Examples of suitable spin columns include QIAquick™ PCR Purification Kit (Qiagen, Valencia, Calif.), and the like. Although the cDNA purification step is optional, purifying the cDNA substantially improves the signal resolution, strength and intensity of the detectable signal in the assay.

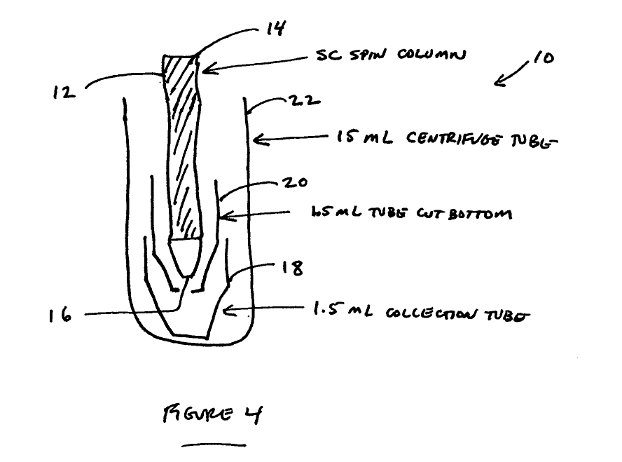

The excess RT primer may be removed via a conventional spin column assembly 10 shown in FIG. 4. The assembly 10 includes a spin column 12 composed of a spin column media 14. The spin column media 14 is composed of a size exclusion resin core, which comprises a plurality of resin pores distributed therethrough. The resin pores are of a suitable size to capture the excess RT primer and permit the cDNA to pass into the void volume. The spin column 12 includes an outlet 16 which is placed into a collection tube 18 and retained in position by a funnel-like piece 20 to prevent the outlet 16 from contacting the bottom of the collection tube 18. The assembly 10 is placed in a centrifuge tube 22 for introduction into a centrifuge apparatus (not shown).

To initiate the purification, the cDNA-containing mixture is placed into a holding tube at the inlet end of the spin column 12 whereupon the spin column 12 and mixture are subjected to high centrifugal force for a sufficient time. The mixture diffuses through the column 12 and exits at the outlet 16 into the collection tube 18. The resulting eluate collected in the tube 18 comprises the purified cDNA which is then used in the microarray assay.

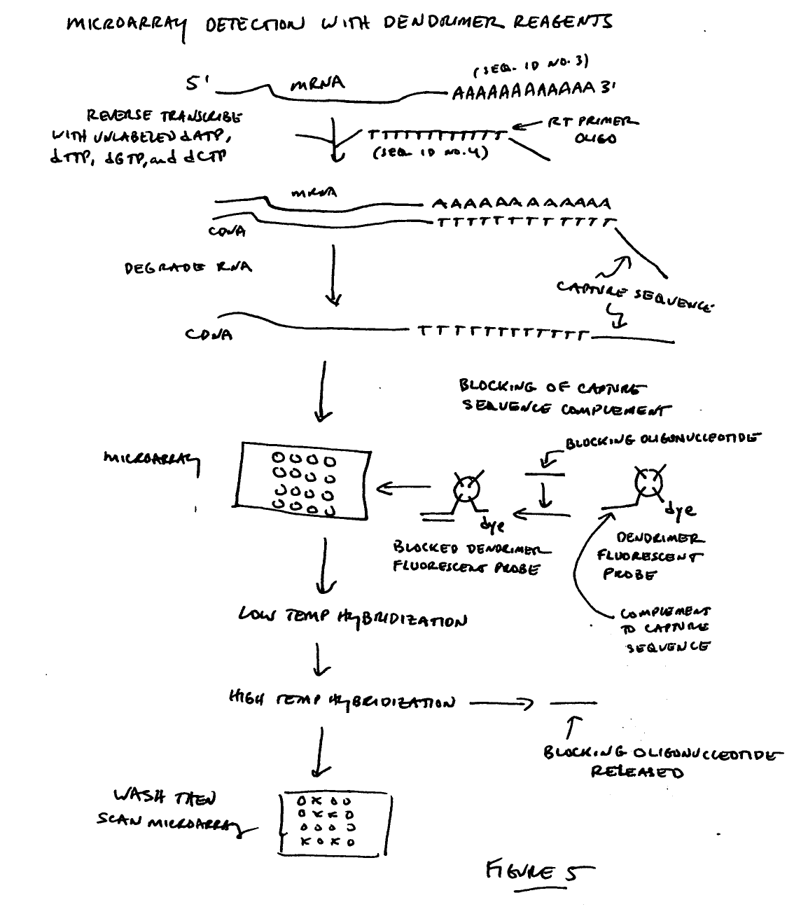

With reference to FIG. 5, an alternate embodiment of the invention of the method is illustrated in which temperature cycling is conducted by raising the temperature from an initial low temperature to a later high temperature. This embodiment, constitutes a “cycling up” of temperature, as opposed to the “cycling down” of temperature previously described. The steps for preparing the target nucleic acid reagent (e.g., cDNA) is the same as described in FIG. 2. In the present embodiment, the capture reagent, preferably in the form of a dendrimer, is prepared using the same process described above. However, in the embodiment of FIG. 5, unlike the embodiment of FIG. 2, the capture sequence complement of the capture reagent is subsequently pre-hybridized to a blocking oligonucleotide to yield a blocked capture reagent. (Or, similarly, in an alternative embodiment, the blocker can be hybridized to the capture sequence of the target nucleic acid, to yield a blocked target nucleic acid and prevent hybridization between the target nucleic acid and the capture reagent).

The blocking oligonucleotide is composed of a portion of the nucleic acid sequence present in the capture sequence of the target nucleic acid reagent (e.g., cDNA). The shorter blocking oligonucleotide has less thermal stability than the full length capture sequence of the cDNA. The melt temperature (Tₘ) of the blocking oligonucleotide is thus lower than the capture sequence. The Tₘ is the temperature at which the bonding between hybridized nucleotides becomes elastic or destabilized, and thus susceptible to separation. Accordingly, paired hybridized nucleotides readily separate at temperatures above the melt temperature of the nucleotides. The pre-hybridization of the capture reagent with the blocking oligonucleotide serves to prevent or at least minimize any unintentional hybridization that could interfere with or obstruct the initial hybridization between the target nucleic acid reagent (e.g., cDNA) and the microarray.

In the present invention, the blocking nucleotide is added to the capture reagent and incubated at a temperature for a time sufficient to facilitate hybridization. Alternatively, this mixture can be preformed during manufacture and can be provided as a component in a kit. Preferably, the blocking oligonucleotide and the capture reagent are incubated at 5° C. below the melt temperature of the blocking oligonucleotide for about 45 minutes in a hybridization buffer. Thereafter, the target nucleic acid reagent and the blocked capture reagent are added to the microarray. The microarray is incubated at a suitable temperature for a sufficient time such that the target nucleic acid reagent is induced to hybridize with the complementary gene probes on the microarray while maintaining the pre-hybridization bonds between the blocking oligonucleotide and the capture reagent. Preferably, the microarray is incubated overnight at a first temperature lower than the melt temperature of the blocking oligonucleotide. More preferably, the microarray is incubated at a temperature 5° C. below the melt temperature of the blocking oligonucleotide. It is to be understood that the melt temperature of an oligonucleotide depends on the length and composition thereof used, which can be determined by those skilled in the art.

Thereafter, the microarray is incubated at a higher second temperature for a time sufficient to induce the blocking oligonucleotide to disassociate from the capture reagent, and facilitate the hybridization of the capture sequence and the target nucleic acid reagent. Preferably, the microarray is incubated at a second temperature of from about 50° C. to 70° C., more preferably about 55° C., for about 3 to 6 hours. (It is to be understood that the temperatures provided in the present application are optimized for the buffer containing approximately 20-30% concentration formamide. With other buffers, other temperatures may be optimal). At the higher temperature, the blocking oligonucleotide becomes displaced, which allows the “unblocked” complement of the capture reagent to hybridize with the capture sequence of the target nucleic acid reagent bound to the microarray. The use of blocking oligonucleotides in the manner described above, substantially improves accuracy of the assay and the signal resolution, strength and intensity of the detectable signal in the assay.

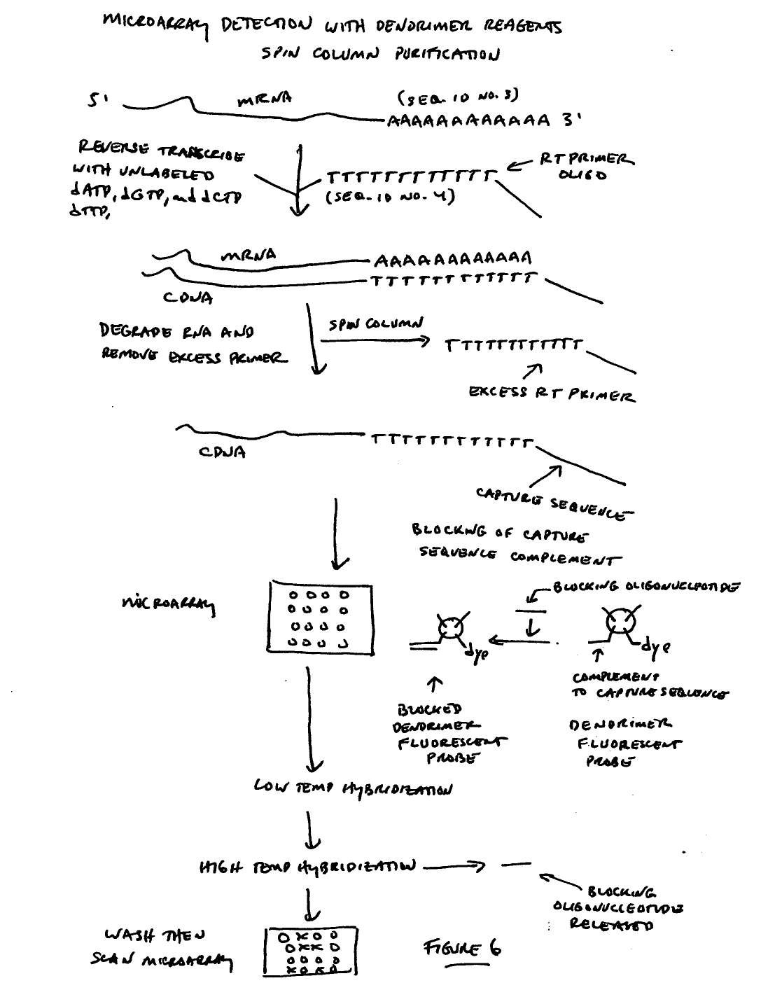

Referring to FIG. 6, a schematic representation of a method of the present invention is illustrated for an alternate embodiment of the present invention. The steps of the method are essentially the same as the method described in FIG. 5 except for the addition of an optional purification step utilizing a spin column to purify the new formed target nucleic acid reagent (e.g., cDNA) prior to its application to the microarray. The spin column purification process is the same as described above

The forgoing discussion discloses and describes merely exemplary embodiments of the present invention. One skilled in the art will readily recognize from such discussion, and from the accompanying claims, that various changes, modifications, and variations can be made therein without departing from the spirit and scope of the invention as defined in the following claims.

### EXAMPLE 1

With reference to FIG. 2, a method for detection and assay on a microarray is described below.

### Microarray Preparation

A microarray was prepared as directed by the manufacturer or by customary procedure protocol. The nucleic acid sequences comprising the DNA or gene probes were amplified using known techniques in polymerase chain reaction, then spotted onto glass slides, and processed according to conventional procedures.

### Preparation and Concentration of Target Nucleic Acid Reagent

The target nucleic acid reagent, or cDNA, was prepared from total RNA or poly(A)+RNA extracted from a sample of cells. It is noted that for samples containing about 10 to 20 μg of total RNA or 500-1000 ng of poly(A)⁺RNA, ethanol precipitation is not required and may be skipped, because the cDNA is sufficiently concentrated to perform the microarray hybridization. In a microfuge tube, 0.25 to 5 μg of total RNA or 12.5 to 500 ng of poly(A)⁺RNA was added with 3 μL of Cy3™ or Cy₅ ™ RT primer (0.2 pmole) (Genisphere, Inc., Montvale, N.J.) and RNase free water for a total volume of 10 μL to yield a RNA-RT primer mixture. The resulting mixture was mixed and microfuged briefly to collect contents in the bottom of the microfuge tube. The collected contents were then heated to 80° C. for about ten (10) minutes and immediately transferred to ice. In a separate microfuge tube on ice, 4 μL of 5X RT buffer, 1 μL of dNTP mix, 4 μL of RNase free water, and 1 μL of reverse transcriptase enzyme (200 Units) were combined to yield a reaction mixture.

The reaction mixture was gently mixed and microfuged briefly to collect contents in the bottom of the microfuge tube. 10 μL of the RNA-RT primer mixture and 10 μL of the reaction mixture, was mixed briefly and incubated at 42° C. for two hours. The reaction was terminated by adding 3.5 μL of 0.5 M NaOH/50mM EDTA to the mixture. The mixture was incubated at 65° C. for ten (10) minutes to denature the DNA/RNA hybrids and the reaction was neutrilized with 5 μL of 1 M Tris-HCl, pH 7.5. 38.5 μL of 10 mM Tris, pH 8.0, 1 mM EDTA was then added to the neutralized reaction mixture. (The above steps may be repeated replacing the 3 μL of Cy3™ RT primer (0.2 pmole) with 3 μL of Cy5™ RT primer (0.2 pmole) for preparing dual channel expression assays whereby the prepared Cy3™ and Cy5™ cDNA mixture are mixed together with 10 μL of 10 Tris, pH 8.0, 1 mM EDTA, to yield a reaction mixture for processing in the following steps.) 2 μL of a carrier nucleic acid (10 mg/mL linear acrylamide) was added to the neutralized reaction mixture for ethanol precipitation. 175 μL of 3 M ammonium acetate was added to the mixture and then mixed. Then, 625 μL of 100% ethanol was added to the resulting mixture. The resulting mixture was incubated at −20° C. for thirty (30) minutes. The sample was centrifuged at an acceleration rate greater than 10,000 g for fifteen (15) minutes. The supematant was aspirated and then 330 μL of 70% ethanol was added to the supematant, or cDNA pellet. The cDNA pellet was then centrifuged at an acceleration rate greater than 10,000 g for 5 minutes, was then remove. The cDNA pellet was dried (i.e., 20-30 minutes at 65° C).

### Hybridization of cDNA/Dendrimer Capture Reagent Mixture to Microarray

The DNA hybridization buffer was thawed and resuspended by heating to 65° C for ten (10) minutes. The hybridization buffer comprised of 40% formamide, 4X SSC, and 1% SDS. The buffer was mixed by inversion to ensure that the components were resuspended evenly. The heating and mixing was repeated until all of the material was resuspended. A quantity of competitor DNA was added as required (e.g. 1 μg of COT-1-DNA, and 0.5 μg of polydT). The cDNA was resuspended in 5.0 μL of sterile water.

In a first embodiment, single channel analysis, 2.5 μL of one type of 3DNA™ reagent (Genisphere, Inc., Montvale, N.J.) (Cy3 or Cy5) comprising the labeled dendrimer capture reagent, was added to the resuspended cDNA along with 12.5 μL of a DNA hybridization buffer (containing 40% formamide). In an alternative embodiment, for dual channel analysis, 2.5 μL of two types of 3DNA™ reagents, Cy3 and Cy5 specifically labeled dendrimer capture reagents, were added to the resuspended cDNA along with 10 μL of a DNA hybridization buffer. In a further embodiment of multiple channel analysis (with three or more channels), 2.5 μL of three or more types of 3DNA™ reagents, Cy3, Cy5, and one or more prepared using another label moiety, were added to the resuspended cDNA along with 10 μL of a DNA hybridization buffer.

For larger hybridization buffer volumes, additional DNA hybridization buffer may be added to the required final volume. It is noted that hybridization buffer volumes greater than 35 μL may also require additional 3DNA™ reagents.

The DNA hybridization buffer mixture was then added to the microarray and then incubated overnight at 65° C. At this stage the cDNA was hybridized to the gene probes and the 3DNA™ reagents remained unbound to the cDNA containing the capture sequence complement. After the overnight hybridization the temperature was cycled down to 50° C. and the hybridization was continued for an additional 4 hours. At this lower temperature the 3DNA™ reagents can now bind to the cDNA that has been bound to the gene probes on the microarray via hybridization of the capture sequence on the 3DNA™ reagent and the complement that is part of the cDNA bound to the gene probe on the microarray.

### Post Hybridization Wash

The microarray was briefly washed to remove any excess dendrimer capture reagents. First, the microarray was washed for 10 minutes at 55° C. with 2X SSC buffer, 0.2% SDS. Then the microarray was washed for 10 minutes at room temperature with 2X SSC buffer. Finally the microarray was washed for 10 minutes at room temperature with 0.2X SSC buffer.

### Signal Detection

The microarray was then scanned as directed by the scanner's manufacturer for detecting, analyzing, and assaying the hybridization pattern.

### EXAMPLE 2

With reference to FIG. 3, a method for detection and assay on a microarray is described below. This method includes the use of a spin column assembly for reducing protocol time and the number of steps for processing and assaying, and for increasing signal strength.

### Microarray Preparation

A microarray was prepared as directed by the manufacturer or by customary protocol procedures. The nucleic acid sequences comprising the DNA or gene probes were amplified using known techniques in polymerase chain reaction, then spotted onto glass slides, and processed according to conventional procedures.

### Preparation and Concentration of Target Nucleic Acid Reagent

The target nucleic acid reagent, or cDNA, was prepared from total RNA or poly(A)+RNA extracted from a sample of cells. In a microfuge tube, 0.25 to 5 μg of total RNA or 12.5 to 500 ng of poly(A)⁺RNA was added with 1 μL of Cy3™ or Cy5™ RT primer (5 pmole) and RNase free water for a total volume of 10 μL to yield a RNA-RT primer mixture. The resulting mixture was mixed and microfuged briefly to collect contents in the bottom of the microfuge tube. The collected contents was then heated to 80° C. for about ten (10) minutes and immediately transferred to ice. In a separate microfuge tube on ice, 4 μL of 5X RT buffer, 1 μL of dNTP mix, 4 μL of RNase free water, and 1 μL of reverse transcriptase enzyme (200 Units) were combined to yield a reaction mixture. The reaction mixture was gently mixed and microfuged briefly to collect contents in the bottom of the microfuge tube. 10 μL of the RNA-RT primer mixture and 10 μL of the reaction mixture was mixed together and incubated at 42° C. for two hours. The reaction was terminated by adding 3.5 μL of 0.5 M NaOH/50 mM EDTA. The mixture was incubated at 65° C. for ten (10) minutes to denature the DNA/RNA hybrids. The reaction was neutralized by the addition of 5 μL of 1 M Tris-HCl, pH 7.5 to the mixture. 71 μL of 10 mM Tris, pH 8.0, 1 mM EDTA was added to the neutralized reaction mixture. (The above steps may be repeated replacing the 1 μL of Cy3™ RT primer (5 pmole) with 1 μL of Cy5™ RT primer (5 pmole) for preparing dual channel expression assays whereby the prepared Cy3™ and Cy5™ cDNA mixture are mixed together with 42 μL of 10 mM Tris, pH 8.0, 1 mM EDTA, to yield a reaction mixture for processing in the following steps.)

### cDNA Purification: Removal of Excess RT Primer via an SC Spin Column Assembly

The spin column was inverted several times to resuspend the media and to create an even slurry in the column. The top and bottom caps were removed from the spin column. A microfuge tube was obtained and the bottom tip of the microfuge tube, was snipped off or punctured. One end of the spin column was placed into the punctured microfuge tube, then the punctured microfuge tube was placed into a second, intact microfuge tube, or collection tube. The assembled spin column was then placed into a 15 mL centrifuge tube with the microfuge tube end first as shown in FIG. 4. The spin column was centrifuged at about 1000 g for about 3.5 minutes after reaching full acceleration. The spin column was checked to ensure that the column was fully drained after centrifugation and that the end of the spin column was above the liquid line in the collection tube. The collection tube contained about 2 to 2.5 mL of clear buffer voided from the spin column. The resin appeared nearly dry in the column barrel, and well packed without distortions or cracks. If the end of the spin column had been immersed in the liquid portion, the spin column would have been discarded and the above steps repeated with a fresh spin column. The spin column was at that point, prepared to remove the excess RT primer in the neutralized reaction mixture.

The drained spin column was removed and a new 1.0 mL collection tube was placed on top of the buffer collection tubes already in the 15 mL centrifuge tube. The voided buffer was discarded. The drained spin column was placed into the new collection tube. 100 μL of the neutralized reaction mixture containing the cDNA was loaded directly into the center of the spin column media The spin column assembly was centrifuged at 10,000x g for about 2.5 minutes upon reaching full acceleration. The eluate collected in the new collection tube was then recovered. About 10 percent of the original reaction mixture was recovered. The eluate comprised the cDNA probe.

2 μL of a carrier nucleic acid (10 mg/mL linear acrylamide) was added to the eluate for ethanol precipitation. 250 μL of 3 M ammonium acetate was added to the mixture and mix. Then, 875 μL of 100% ethanol was added to the mixture. The resulting mixture was incubated at −20° C. for thirty (30) minutes. The sample was centrifuged at an acceleration rate greater than 10,000x g for fifteen (15) minutes. The supernatant was aspirated and 300 μL of 70% ethanol was added to the supernatant, or the cDNA pellet. The cDNA pellet was then centrifuged at an acceleration rate greater than 10,000x g for 5 minutes. The supernatant was then removed. The cDNA pellet was dried (i.e. 20-30 minutes at 65° C.).

### Hybridization of cDNA/Dendrimer Capture Reagent Mixture to Microarray

The DNA hybridization buffer was thawed and resuspended by heating to 65° C. and maintained at 65° C. for ten (10) minutes. The hybridization buffer comprised of 40% formamide, 4X SSC, and 1% SDS. The buffer was mixed by inversion to ensure that the components were resuspended evenly. The heating and mixing was repeated until all the material was resuspended. A quantity of competitor DNA (e.g. 1.0 μg of COT-1-DNA, and 0.5 μg of polydT) may be added, if required. The cDNA was resuspended in 5.0 μL of sterile water.

In a first embodiment, single channel analysis, 2.5 μL of one type of 3DNA™ reagent (Genisphere, Inc., Montvale, N.J.) (Cy3 or Cy5) was added to the resuspended cDNA along with 12.5 μL of a DNA hybridization buffer (containing 40% formamide). In an alternative embodiment, for dual channel analysis, 2.5 μL of two types of 3DNA™ reagents, Cy3 and Cy5 specifically labeled dendrimer capture reagents, were added to the resuspended cDNA along with 10 μL of a DNA hybridization buffer. In a further embodiment of multiple channel analysis (with three or more channels), 2.5 μL of three or more types of 3DNA™ reagents, Cy3, Cy5, and one or more prepared using another label moiety, were added to the resuspended cDNA along with 10 μL of a DNA hybridization buffer.

For larger hybridization buffer volumes, additional amounts of the DNA hybridization buffer may be added to reach the required final volume. It is also noted that hybridization buffer volumes greater than 35 μL may also require additional 3DNA™ reagents.

The DNA hybridization buffer mixture was then added to the microarray and then incubated overnight at 65° C. At this stage the cDNA was hybridized to the gene probes and the 3DNA™ reagents remained unbound to the cDNA containing the capture sequence complement. After the overnight hybridization the temperature was cycled down to 50° C. and the hybridization was continued for an additional 4 hours. At this lower temperature the 3DNA™ reagents can now bind to the cDNA that has been bound to the gene probes on the microarray via hybridization of the capture sequence on the 3DNA™ reagent and the complement that is part of the cDNA bound to the gene probe on the microarray.

### Post Hybridization Wash

The microarray was briefly washed to remove any excess dendrimer capture reagents. First, the microarray was washed for 10 minutes at 55° C. with 2X SSC buffer, containing 0.2% SDS. Then, the microarray was washed for 10 minutes at room temperature with 2X SSC buffer. Finally, the microarray was washed for 10 minutes at room temperature with 0.2X SSC buffer.

### Signal Detection

The microarray was then scanned as directed by the scanner's manufacturer for detecting, analyzing, and assaying the hybridization pattern.

### EXAMPLE 3

With reference to FIG. 4 , a method for detection and assay on a microarray is described below. This method includes a step and oligonucleotide reagents that result in the blocking of the capture sequence complement on the 3DNA™ reagent with an oligonucleotide, blocking oligonucleotide, whose sequence is equal to a portion of the capture sequence that is part of the primer. As a result of the design of this oligonucleotide, the melting temperature is lower than that of the full length capture sequence. Thus, upon cycling the temperature of hybridization from a temperature at or below that of the blocking oligonucleotide to a temperature greater than that of the blocking nucleotide but less than that of the capture sequence, the blocking oligonucleotide will be displaced for the 3DNA™ reagent and to be replaced by the capture sequence that is part of the cDNA primer.

### Microarray Preparation

A microarray was prepared as directed by the manufacturer or by customary procedure protocol. The nucleic acid reagent comprising the DNA or gene probes were amplified using known techniques in polymerase chain reaction, then spotted onto glass slides, and processed according to conventional procedures.

### Preparation and Concentration of Target Nucleic Acid Reagent

The target nucleic acid reagent, or cDNA, was prepared from total RNA or poly(A)+RNA extracted from a sample of cells. It is noted that for samples containing about 10 to 20 μg of total RNA or 500-1000 ng of poly(A)⁺RNA, ethanol precipitation is not required and may be skipped, because the cDNA is sufficiently concentrated to perform the microarray hybridization. In a microfuge tube, 0.25 to 5 μg of total RNA or 12.5 to 500 ng of poly(A)⁺RNA was added with 3 μL of Cy3™ or Cy5™ RT primer (0.2 pmole) and RNase free water for a total volume of 10 μL to yield a RNA-RT primer mixture. The resulting mixture was mixed and microfuged briefly to collect contents in the bottom of the microfuge tube. The collected contents were then heated to 80° C. for about ten (10) minutes and immediately transferred to ice. In a separate microfuge tube on ice, 4 μL of 5X RT buffer, 1 μL of dNTP mix, 4 μL of RNase free water, and 1 μL of reverse transcriptase enzyme (200 Units) were combined to yield a reaction mixture. The reaction mixture was gently mixed and microfuged briefly to collect contents in the bottom of the microfuge tube. 10 μL of the RNA-RT primer mixture and 10 μL of the reaction mixture, was mixed briefly and incubated at 42° C. for two hours. The reaction was terminated by adding 3.5 μL of 0.5 M NaOH/50 mM EDTA to the mixture. The mixture was incubated at 65° C. for ten (10) minutes to denature the DNA/RNA hybrids and the reaction was neutralized with 5 μL of 1 M Tris-HCl, pH 7.5. 38.5 μL of 10 mM Tris, pH 8.0, 1 mM EDTA was then added to the neutralized reaction mixture. (The above steps may be repeated replacing the 3 μL of Cy3™ RT primer (0.2 pmole) with 3 μL of Cy5™ RT primer (0.2 pmole) for preparing dual channel expression assays whereby the prepared Cy3™ and Cy5™ cDNA mixture are mixed together with 10 μL of 10 Tris, pH 8.0, 1 mM EDTA, to yield a reaction mixture for processing in the following steps.)

2 μL of a carrier nucleic acid (10 mg/mL linear acrylamide) was added to the neutralized reaction mixture for ethanol precipitation. 175 μL of 3 M ammonium acetate was added to the mixture and then mixed. Then, 625 μL of 100% ethanol was added to the resulting mixture. The resulting mixture was incubated at −20° C. for thirty (30) minutes. The sample was centrifuged at an acceleration rate greater than 10,000 g for fifteen (15) minutes. The supernatant was aspirated and then 330 μL of 70% ethanol was added to the supernatant, or cDNA pellet. The cDNA pellet was then centrifuged at an acceleration rate greater than 10,000 g for 5 minutes, was then remove. The cDNA pellet was dried (i.e., 20-30 minutes at 65° C.).

### Hybridization of cDNA/Dendrimer Capture Reagent Mixture to Microarray

The DNA hybridization buffer was thawed and resuspended by heating to 65° C. for ten (10) minutes. The hybridization buffer comprised of 40% formamide, 4X SSC, and 1% SDS. The buffer was mixed by inversion to ensure that the components were resuspended evenly. The heating and mixing was repeated until all of the material was resuspended. A quantity of competitor DNA was added as required (e.g. 1 μg of COT-1-DNA, and 0.5 μg of polydT). The cDNA was resuspended in 5.0 μL of sterile water.

### Blocking of 3DNA™ Reagent Capture Sequences

In a first embodiment, single channel analysis, 2.5 μL of one type of 3DNA™ reagent (Genisphere, Inc., Montvale, N.J.) (Cy3 or Cy5) and 1 μL (450 femptomoles) of the blocking oligonucleotide mixture were added to 12.5 μL of a DNA hybridization buffer (containing 40% formamide). In an alternative embodiment, for dual channel analysis, 2.5 μL of two types of 3DNA™ reagents, Cy3 and Cy5 specifically labeled dendrimer capture reagents, and 1 μL of the blocking oligonucleotide (450 femptomoles of each corresponding oligonucleotide) were added to 10 μL of a DNA hybridization buffer. In a further embodiment of multiple channel analysis (with three or more channels), 2.5 μL of three or more types of 3DNA™ reagents, Cy3, Cy5, and one or more prepared using another label moiety, and 1 μL of the blocking oligonucleotide (450 femptomoles of each corresponding oligonucleotide) were added to 10 μL of a DNA hybridization buffer of multiple channel analysis (with three or more channels), 2.5 μL of three or more types of 3DNA™ reagents, Cy3, Cy5, and one or more prepared using another label moiety, were added to the resuspended cDNA along with 10 μL of a DNA hybridization buffer.

The mixture was incubated at 5° C. below the approximate Tm of the blocking oligonucleotide (37° C.) in this hybridization buffer for 45 minutes. After 45 minutes, all of this material was added to the resuspended cDNA.

For larger hybridization buffer volumes, additional DNA hybridization buffer may be added to the required final volume. It is noted that hybridization buffer volumes greater than 35 μL may also require additional 3DNA™ reagents.

The DNA hybridization buffer mixture was then added to the microarray and then incubated overnight at 5° C. below the approximate Tm of the blocking oligonucleotide (37° C.). At this stage the cDNA was hybridized to the gene probes and the 3DNA™ reagents remained unbound to the cDNA containing the capture sequence complement. After the overnight hybridization the temperature was cycled up to 55° C. and the hybridization was continued for an additional 4 hours. At this higher temperature the blocking oligonucleotide becomes displaced and the 3DNA™ reagents can now bind to the cDNA that has been bound to the gene probes on the microarray via hybridization of the capture sequence on the 3DNA™ reagent and the complement that is part of the cDNA bound to the gene probe on the microarray.

### Post Hybridization Wash

The microarray was briefly washed to remove any excess dendrimer capture reagents. First, the microarray was washed for 10 minutes at 55° C. with 2X SSC buffer, 0.2% SDS. Then the microarray was washed for 10 minutes at room temperature with 2X SSC buffer. Finally the microarray was washed for 10 minutes at room temperature with 0.2X SSC buffer.

### Signal Detection

The microarray was then scanned as directed by the scanner's manufacturer for detecting, analyzing, and assaying the hybridization pattern.

### EXAMPLE 4

With reference to FIG. 5, a method for detection and assay on a microarray is described below. This method includes the use of a spin column assembly for reducing protocol time and number of steps, and for increasing signal strength. This method also includes a step and oligonucleotide reagents that result in the blocking of the capture sequence complement on the 3DNA™ reagent with an oligonucleotide, blocking oligonucleotide, whose sequence is equal to a portion of the capture sequence that is part of the primer. As a result of the design of this oligonucleotide, the melting temperature is lower than that of the full length capture sequence. Thus upon cycling the temperature of hybridization form a temperature at or below that of the blocking oligonucleotide to a temperature greater than that of the blocking nucleotide but less than that of the capture sequence, the blocking oligonucleotide will be displaced for the 3DNA™ reagent and to be replaced by the capture sequence that is part of the cDNA primer.

### Microarray Preparation

A microarray was prepared as directed by the manufacturer or by customary protocol procedures. The nucleic acid reagent comprising the DNA or gene probes were amplified using known techniques in polymerase chain reaction, then spotted onto glass slides, and processed according to conventional procedures.

### Preparation and Concentration of Target Nucleic Acid Reagent

The target nucleic acid reagent, or cDNA was prepared from total RNA or poly(A)+RNA extracted from a sample of cells. In a microfuge tube, 0.25 to 5 μg of total RNA or 12.5 to 500 ng of poly(A)⁺RNA was added with 1 μL of Cy3™ or Cy5™ RT primer (5 pmole) and RNase free water for a total volume of 10 μL to yield a RNA-RT primer mixture. The resulting mixture was mixed and microfuged briefly to collect contents in the bottom of the microfuge tube. The collected contents was then heated to 80° C. for about ten (10) minutes and immediately transferred to ice. In a separate microfuge tube on ice, 4 μL of 5X RT buffer, 1 μL of dNTP mix, 4 μL of RNase free water, and 1 μL reverse transcriptase enzyme (200 Units) were combined to yield a reaction mixture. The reaction mixture was gently mixed and microfuged briefly to collect contents in the bottom of the microfuge tube. 10 μL of the RNA-RT primer mixture and 10 μL of the reaction mixture was mixed together and incubated at 42° C. for two hours. The reaction was terminated by adding 3.5 μL of 0.5 M NaOH/50 mM EDTA. The mixture was incubated at 65° C. for ten (10) minutes to denature the DNA/RNA hybrids. The reaction was neutralized by the addition of 5 μL of 1 M Tris-HCl, pH 7.5 to the mixture. 71 μL of 10 mM Tris, pH 8.0, 1 mM EDTA was added to the neutralized reaction mixture. (The above steps may be repeated replacing the 1 μL of Cy3™ RT primer (5 pmole) with 1 μL of Cy5™ RT primer (5 pmole) for preparing dual channel expression assays whereby the prepared Cy3™ and Cy5™ cDNA mixture are mixed together with 42 μL of 10 mM Tris, pH 8.0, 1 mM EDTA, to yield a reaction mixture for processing in the following steps.)

### cDNA Purification: Removal of Excess RT Primer via a SC Spin Column Assembly

The spin column was inverted several times to resuspend the media and to create an even slurry in the column. The top and bottom caps were removed from the spin column. A microfuge tube was obtained and the bottom tip of the microfuge tube, was snipped off or punctured. One end of the spin column was placed into the punctured microfuge tube, then the punctured microfuge tube was placed into a second, intact microfuge tube, or collection tube. The assembled spin column was then placed into a 15 mL centrifuge tube with the microfuge tube end first as shown in FIG. 4. The spin column was centrifuged at about 1000 g for about 3.5 minutes after reaching full acceleration. The spin column was checked to ensure that the column was fully drained after centrifugation and that the end of the spin column was above the liquid line in the collection tube. The collection tube contained about 2 to 2.5 mL of clear buffer voided from the spin column. The resin appeared nearly dry in the column barrel, and well packed without distortions or cracks. If the end of the spin column had been immersed in the liquid portion, the spin column would have been discarded and the above steps repeated with a fresh spin column. The spin column was at that point, prepared to remove the excess RT primer in the neutralized reaction mixture.

The drained spin column was removed and a new 1.0 mL collection tube was placed on top of the buffer collection tubes already in the 15 mL centrifuge tube. The voided buffer was discarded. The drained spin column was placed into the new collection tube. 100 μL of the neutralized reaction mixture containing the cDNA was loaded directly into the center of the spin column media. The spin column assembly was centrifuged at 10,000x g for about 2.5 minutes upon reaching full acceleration. The eluate collected in the new collection tube was then recovered. About 10 percent of the original reaction mixture was recovered. The eluate comprised the cDNA probe.

2 μL of a carrier nucleic acid (10 mg/mL linear acrylamide) was added to the eluate for ethanol precipitation. 250 μL of 3 M ammonium acetate was added to the mixture and mix. Then, 875 μL of 100% ethanol was added to the mixture. The resulting mixture was incubated at −20° C. for thirty (30) minutes. The sample was centrifuged at an acceleration rate greater than 10,000x g for fifteen (15) minutes. The supernatant was aspirated and 300 μL of 70% ethanol was added to the supernatant, or the cDNA pellet. The cDNA pellet was then centrifuged at an acceleration rate greater than 10,000x g for 5 minutes. The supernatant was then removed. The cDNA pellet was dried (i.e. 20-30 minutes at 65° C.).

### Hybridization of cDNA/Dendrimer Capture Reagent Mixture to Microarray

The DNA hybridization buffer was thawed and resuspended by heating to 65° C. and maintained at 65° C. for ten (10) minutes. The hybridization buffer comprised of 40% formamide, 4X SSC, and 1% SDS. The buffer was mixed by inversion to ensure that the components were resuspended evenly. The heating and mixing was repeated until all the material was resuspended. A quantity of competitor DNA (e.g. 1.0 μg of COT-1-DNA, and 0.5 μg of polydT) may be added, if required. The cDNA was resuspended in 5.0 μL of sterile water.

### Blocking of 3DNA™ Reagent Capture Sequences

In a first embodiment, single channel analysis, 2.5 μL of one type of 3DNA™ reagent (Genisphere, Inc., Montvale, N.J.) (Cy3 or Cy5) and 1 μL (450 femptomoles) of the blocking oligonucleotide mixture were added to 12.5 μL of a DNA hybridization buffer (containing 40% formamide). In an alternative embodiment, for dual channel analysis, 2.5 μL of two types of 3DNA™ reagents, Cy3 and Cy5 specifically labeled dendrimer capture reagents, and 1 μL of the blocking oligonucleotide L (450 femptomoles of each corresponding oligonucleotide) were added to 10 μL of a DNA hybridization buffer. In a further embodiment of multiple channel analysis (with three or more channels), 2.5 μL of three or more types of 3DNA™ reagents, Cy3, Cy5, and one or more prepared using another label moiety, and 1 μL of the blocking oligonucleotide (450 femptomoles of each corresponding oligonucleotide) were added to 10 μL of a DNA hybridization buffer of multiple channel analysis (with three or more channels), 2.5 μL of three or more types of 3DNA™ reagents, Cy3, Cy5, and one or more prepared using another label moiety, were added to the resuspended cDNA along with 10 μL of a DNA hybridization buffer. The mixture was incubated at 5° C. below the approximate Tm of the blocking oligonucleotide (37° C.) in this hybridization buffer for 45 minutes. After 45 minute all of this material was added to the resuspended cDNA.

For larger hybridization buffer volumes, additional DNA hybridization buffer may be added to the required final volume. It is noted that hybridization buffer volumes greater than 35 μL may also require additional 3DNA™ reagents.

The DNA hybridization buffer mixture was then added to the microarray and then incubated overnight at 5° C. below the approximate Tm of the blocking oligonucleotide (37° C.). At this stage the cDNA was hybridized to the gene probes and the 3DNA™ reagents remained unbound to the cDNA containing the capture sequence complement. After the overnight hybridization the temperature was cycled up to 55° C. and the hybridization was continued for an additional 4 hours. At this higher temperature the blocking oligonucleotide is displaced and the 3DNA™ reagents can now bind to the cDNA that has been bound to the gene probes on the microarray via hybridization of the capture sequence on the 3DNA™ reagent and the complement that is part of the cDNA bound to the gene probe on the microarray.

### Post Hybridization Wash

The microarray was briefly washed to remove any excess dendrimer capture reagents. First, the microarray was washed for 10 minutes at 55° C. with 2X SSC buffer, containing 0.2% SDS. Then, the microarray was washed for 10 minutes at room temperature with 2X SSC buffer. Finally, the microarray was washed for 10 minutes at room temperature with 0.2X SSC buffer.

### Signal Detection

The microarray was then scanned as directed by the scanner's manufacturer for detecting, analyzing, and assaying the hybridization pattern.

### EXAMPLE 5

### Spin Column Assembly Procedure

A method for determining the presence of a specific sequence of nucleotides in a nucleic acid target molecule sample on a microarray further utilizing a spin column assembly is described below. The procedures for preparing the microarray and labeled dendrimer capture reagents are the same as described in Examples 1-4.

### Preparation and Concentration of Target Nucleic Acid Reagent

The target nucleic acid reagent was prepared from total RNA extracted from a sample of cells using standard methods. For this particular example the portion of the total RNA population comprising that to be known as poly(A)⁺RNA also commonly referred to as messenger RNA (mRNA) was used as a template for producing the target nucleic acid reagent in the form of cDNA. About 0.25 to 1 μg of input total RNA or 12.5 to 50 ng of poly(A)⁺RNA was extracted and isolated using known methods. 3μl of Cy3™ and/or Cy5™ RT Primer Oligo (0.2 pmole) were each obtained. For single channel analysis, only one RT primer was used. For dual channel analysis, multiple RT primers were used. The RT primers used included the following capture sequences:

Cy3™ RT primer capture sequence: 5′-ggC Cga CTC ACT gCg CgT CTT CTg TCC CgC C-3′; and

Cy5™ RT primer capture sequence: 5′-CCT gTT gCT CTA TTT CCC gTg CCg CTC Cgg T-3.

The RNA and RT primer was added to RNase-free water in a microtube to yield a RNA-RT primer mix with a final volume of about 10 μl. The mix was briefly microfuged to collect the contents to the bottom of the microtube and then heated to 80° C. for about 10 minutes. The microtube was immediately put into an ice bath. In a separate microtube, 4 μl of 5X RT buffer, 4 μl dNTP mix, 4 μl of RNase-free water, 1 ml of reverse transcriptase enzyme (200 Units) to yield about 10 μl of a reaction mix. The reaction mix was gently mixed and briefly microfuged to collect the contents to the bottom of the tube. The RNA-RT primer mix and the reaction mix were mixed together and incubated at 42° C. for about 2 hours. The reaction was stopped by adding 3.5 μl of 0.5 M NaOH/50 mM Tris-HCl, pH 7.5.

In a first embodiment, for single channel analysis, 71 μl of 10 mM Tris, pH 8.0, 1 mM EDTA was added to the resulting mixture. In an alternative embodiment, for dual channel analysis, the mixture containing Cy3™ cDNA was combined with the mixture containing Cy5™ cDNA and mixed with 42 μl of 10 mM Tris, pH 8.0, 1 mM EDTA.

a.) Spin Column Purification A spin column (QIAquick™ PCR Purification Kit) was obtained and inverted several times to resuspend the media and to create an even slurry in the column. The top cap and bottom cap of the spin column was removed. A microfuge tube was obtained with the bottom tip cut off. The column was placed into the modified tube. The modified tube was placed into an intact microfuge tube. The entire construction was placed into a 15 ml centrifuge tube with the intact microfuge tube at the bottom to yield the assembly shown in FIG. 4.

The assembly was centrifuged at 1000 g for about 3.5 minutes or until the column was fully drained. It is critical that the bottom of the spin column does not contact the drained liquid in the microfuge tube. If contact occurs, the spin column must be discarded and the above steps for preparing the spin column repeated.

The cDNA mixture was loaded into the center of the column media and centrifuged at 1000 g for about 2.5 minutes. The eluate containing the purified cDNA was collected. The volume was about 10% of the original volume loaded into the spin column.

b.) Ethanol Precipitation

2 μl of carrier nucleic acid (10 mg/ml tRNA or 1 μg/μl CoT1 DNA) and 250 μ1 of 3 M ammonium acetate were added to the eluate and mixed. Then 875 μl of 100% ethanol was added to the mixture and incubated at −20° C. for about 30 minutes. The mixture was then centrifuged at 10,000 g for about 10 minutes. The supernatant was aspirated. 300 μl of 70% ethanol was added to the cDNA pellet. The mixture was centrifuged at 10,000 g for about 5 minutes. The supernatant was removed. The cDNA pellet was dried at 65° C. for about 20 to 30 minutes.

The procedural steps for the hybridizations of the cDNA, labeled dendrimer capture reagent, and the microarray, post hybridization wash, and signal detection are then conducted as in Examples 1-4.

### EXAMPLE 6

### Alternate Procedure for the Preparation and Concentration of cDNA

For quantities of total RNA in the range of from about 5 to 10 μg and of poly(A)⁺RNA in the range of from about 250 to 500 ng, ethanol precipitation was not required. The cDNA produced from RNA in such quantities was present in a sufficient concentration to implement the microarray hybridization without the use of ethanol precipitation.

About 5 to 10 μg of input total RNA or 250 to 500 ng of poly(A)⁺RNA was extracted and isolated using known methods. 1 μl of RT primer (5 pmole) was mixed with the RNA. For single channel analysis, only one RT primer was used. For dual channel analysis, multiple RT primers were used. The RT primers used included the following capture sequences:

Cy3 ™ RT primer capture sequence:
5′-ggC Cga CTC ACT gCg CgT CTT CTg TCC CgC C -3′;
and
Cy5 ™ RT primer capture sequence:
5′-CCT gTT gCT CTA TTT CCC gTg CCg CTC Cgg T -3.

The RNA and RT primer were added to RNase-free water in a microtube to yield a RNA-RT primer mix with a final volume of about 10 μl. The mix was briefly microfuged to collect the contents to the bottom of the microtube and then heated to 80° C. for about 10 minutes. The microtube was immediately put into an ice bath.

In a separate microtube, 4 μl of 5X RT buffer, 4 μl dNTP mix, 4 μl of RNase-free water, 1 ml of reverse transcriptase enzyme (200 Units) were mixed to yield about 10 μl of a reaction mix. The reaction mix was gently mixed and briefly microfuged to collect the contents to the bottom of the tube. The RNA-RT primer mix and the reaction mix were mixed together and incubated at 42° C. for about 2 hours. The reaction was stopped by adding 3.5 μl of 0.5 M NaOH/50 mM EDTA. The mixture was incubated at 65° C. for about 10 minutes to denature the DNA/RNA hybrids. The reaction was neutralized with 5 μl of 1 M Tris-HCl, pH 7.5.

In a first embodiment, for single channel analysis, 71 μl of 10 mM Tris, pH 8.0, 1 mM EDTA was added to the resulting mixture. In an alternative embodiment, for dual channel analysis, the mixture containing Cy3™ cDNA was combined with the mixture containing Cy5™ cDNA, and mixed with 42 μl of 10 mM Tris, pH 8.0, 1 mM EDTA.

The resulting mixture was purified using the spin column procedure described in Example 5. The aliquot of the eluate was then utilized in the microarray hybridization procedure described in Examples 1-4.

Having described this invention with regard to specific embodiments, it is to be understood that the description is not meant as a limitation since further embodiments, modifications and variations may be apparent or may suggest themselves to those skilled in the art It is intended that the present application cover all such embodiments, modifications and variations.

1. A method for determining the presence of at least one specific nucleotide sequence in a target nucleic acid reagent extracted from a biological sample, said method comprising the steps of:
(a) concurrently contacting a microarray with:
(i) a target nucleic acid reagent, said target nucleic acid reagent having a nucleotide sequence, said nucleotide sequence further including a capture sequence, and
(ii) a capture reagent, said capture reagent having at least one first arm having a label capable of emitting a detectable signal and at least one second arm having a nucleotide sequence complementary to said capture sequence of said target nucleic acid reagent;
said microarray having thereon a plurality of features, each of said plurality of features including a probe nucleotide sequence; and
(b) treating the microarray from step (a) at a temperature and for a time sufficient to induce said nucleotide sequence of said target nucleic acid to hybridize to the probe nucleotide sequence complementary thereto on the microarray, and to induce said capture reagent to hybridize to said capture sequence of said nucleotide sequence of said target nucleic acid hybridized to the microarray.

2. The method of claim 1, wherein the presence of the latter hybridization results in the emission of the detectable signal from the corresponding feature, and the absence thereof results in no emission of the detectable signal from the corresponding feature, thus generating a detectable hybridization patter for subsequent analysis.

3. The method of claim 1 wherein the capture reagent is selected from the group consisting of dendrimers, carbohydrates, proteins, and nucleic acids.

4. The method of claim 1 wherein the target nucleic acid reagent is cDNA.

5. The method of claim 1 wherein the capture reagent is a dendrimer.

6. The method of claim 1 wherein step (b) further comprises incubating the microarray from step (a) at a first temperature for a first period of time, and thereafter incubating the microarray from step (a) at a lower second temperature for a second period of time which may be different than the first period of time.

7. The method of claim 6 wherein said first temperature and said first period are suitable for hybridization of the target nucleic acid reagent to the probe.

8. The method of claim 6 wherein said second temperature and said second period are suitable for hybridization of the capture reagent to the target nucleic acid reagent.

9. The method of claim 6 wherein the first temperature is in the range of from about 65° C. to 75° C., and the second temperature is in the range of from about 50° to 55° C.

10. The method of claim 6 wherein the first period of time is overnight, and the second period of time is 4 to 6 hours.

11. The method of claim 1 wherein step (b) further comprises incubating the microarray from step (a) at about a temperature in the range from about 65° C. to 75° C. overnight, and thereafter incubating the microarray from step (a) at a temperature in the range of from about 50 to 55° C. for about 4 to 6 hours.

12. The method of claim 1 further comprising forming a mixture of the at least one specific nucleotide sequence of the target nucleic acid reagent and the capture reagent and contacting the microarray with said mixture.

13. The method of claim 1 further comprising the step of utilizing a blocking nucleotide prior to step (a) to block the hybridization of said capture sequence of said target nucleic acid reagent to said capture reagent.

14. The method of claim 1 further comprising the step of pre-hybridizing a blocking nucleotide to the capture reagent prior to step (a) to prevent hybridization between said capture reagent and said capture sequence of said target nucleic acid reagent.

15. The method of claim 1 further comprising the step of pre-hybridizing a blocking nucleotide to the capture sequence of said target nucleic acid reagent prior to step (a) to prevent hybridization between said capture sequence of said target nucleic acid reagent and said capture reagent.

16. The method of claim 1 wherein step (b) further comprises incubating the microarray from step (a) at a first temperature for a first period of time, and thereafter incubating the microarray from step (a) at a higher second temperature for a second period of time which may be different than the first period of time.

17. The method of claim 13 wherein step (b) further comprises incubating the microarray from step (a) at a first temperature for a first period of time, and thereafter incubating the microarray from step (a) at a higher second temperature for a second period of time which may be different than the first period of time.

18. The method of claim 16 wherein said first temperature and said first period are suitable to allow hybridization of the target nucleic acid reagent to the probe.

19. The method of claim 16 wherein said second temperature and said second period are suitable to allow hybridization of the capture reagent to the target nucleic acid reagent.

20. The method of claim 17 wherein the first temperature is below the melt temperature of the blocking oligonucleotide, and the second temperature is at least 5 degrees above the melt temperature of the blocking oligonucleotide yet is also a temperature suitable for binding of the capture reagent to the target nucleic acid.

21. The method of claim 17 wherein the first period of time is overnight, and the second period of time is about 3-5 hours.

22. The method of claim 17 wherein the first temperature is at least 5° C. below the melt temperature of the blocking oligonucleotide.

23. The method of claim 12 wherein step (b) further comprises incubating the microarray from step (a) at the first temperature of about 32° C. overnight, and the incubating the microarray from step (a) at the second temperature of about 55° C. for about 4 hours.

24. The method of claim 1 further comprising the step of utilizing a spin column to prepare said target nucleic acid reagent, prior to step (a).


SEQUENCE LISTING (4 sequences)

SEQ ID NO: 1 - DNA, 48 bp, Artificial Sequence
  Sequence of primer for use with Cy3
ggccgactcactgcgcgtcttctgtcccgccttttttttttttttttt

SEQ ID NO: 2 - DNA, 48 bp, Artificial Sequence
  Sequence of primer for use with Cy5
cctgttgctctatttcccgtgccgctccggtttttttttttttttttt

SEQ ID NO: 3 - RNA, 12 nt, Saccharomyces cerevisiae
  polyA_site (1)..(12)
aaaaaaaaaaaa

SEQ ID NO: 4 - DNA, 12 bp, Artificial Sequence
  Poly T for hybridization to Poly A tail
tttttttttttt